(19) United States
(12) Patent Application Publication
(10) Pub. No.: US 2021/0219897 A1
(43) Pub. Date: Jul. 22, 2021

(54) METHOD AND APPARATUS FOR ANALYZING ELECTRICAL CHARACTERISTICS OF NERVES

(71) Applicant: SAMSUNG ELECTRONICS CO., LTD., Suwon-si (KR)

(72) Inventors: Kyuil LEE, Incheon (KR); Hoyoung AHN, Suwon-si (KR); Munbo SHIM, Yongin-si (KR)

(73) Assignee: SAMSUNG ELECTRONICS CO., LTD., Suwon-si (KR)

(21) Appl. No.: 17/138,093

(22) Filed: Dec. 30, 2020

(30) Foreign Application Priority Data
Jan. 17, 2020 (KR) 10-2020-0006742

(51) Int. Cl. A61B 5/294; A61B 5/00; A61B 5/311; A61B 5/388

(52) U.S. Cl. CPC A61B 5/294; A61B 5/4041; A61B 5/0033; A61B 5/388; A61B 5/311

(57) ABSTRACT

Provided is a method of analyzing electrical characteristics of nerves, the method including generating an input electrical signal to be applied to a nerve, obtaining an output electrical signal based on measuring a nerve signal generated from the nerve in response to the input electrical signal, obtaining output frequency components, which are frequency components of the output electrical signal, based on converting the output electrical signal into a frequency domain, and obtaining conductance of the nerves and capacitance of the nerves based on the output frequency components.

### CROSS-REFERENCE TO RELATED APPLICATION

This application claims priority to Korean Patent Application No. 10-2020-0006742, filed on Jan. 17, 2020, in the Korean Intellectual Property Office, the disclosure of which is incorporated herein in its entirety by reference.

### BACKGROUND

### 1. Field

Example embodiments of the present disclosure relate to a method and apparatus for analyzing electrical characteristics of nerves.

### 2. Description of Related Art

Electrical impedance tomography (EIT) is a technique for imaging electrical characteristics inside a living body. EIT is a non-invasive imaging technique and may be used to image the electrical characteristics of a nerve passing through nerves. By measuring a nerve signal generated when an electrical signal is applied to a nerve, the electrical characteristics of the nerve may be analyzed.

There is a need for a technique capable of analyzing the electrical characteristics of a nerve even when an electrical signal having a high carrier frequency is applied to the nerve.

### SUMMARY

One or more example embodiments provide a method and apparatus for analyzing electrical characteristics of a nerve. The technical objects to be achieved by one or more example embodiments are not limited to the technical objects as described above, and other technical problems may be inferred from the following example embodiments.

Additional aspects will be set forth in part in the description which follows and, in part, will be apparent from the description, or may be learned by practice of example embodiments.

According to an aspect of an example embodiment, there is provided a method of analyzing electrical characteristics of nerves, the method including generating an input electrical signal to be applied to a nerve, obtaining an output electrical signal based on measuring a nerve signal generated from the nerve in response to the input electrical signal, obtaining output frequency components, which are frequency components of the output electrical signal, based on converting the output electrical signal into a frequency domain, and obtaining conductance of the nerves and capacitance of the nerves based on the output frequency components.

The obtaining of the output electrical signal may include obtaining the output electrical signal based on sampling the nerve signal at a sampling frequency, the sampling frequency being smaller than a carrier frequency of the input electrical signal.

The obtaining of the output electrical signal may include obtaining the output electrical signal based on sampling the nerve signal at a sampling frequency, and the sampling frequency may be proportional to a number of the output frequency components.

The obtaining of the output electrical signal may include obtaining, from a carrier frequency of the input electrical signal, the output frequency components corresponding to frequencies included in a predetermined range.

The obtaining of the output electrical signal may include obtaining the output frequency components corresponding to frequencies included in a range from −3 kHz to 3 kHz from a carrier frequency of the input electrical signal.

The obtaining of the conductance may include obtaining frequency components of the conductance, and obtaining the capacitance may include obtaining a value of the capacitance based on the output frequency components.

The obtaining of the conductance and the capacitance may include obtaining input frequency components, which are frequency components of the input electrical signal corresponding to the output frequency components, obtaining an inverse matrix of a matrix including values of the output frequency components as elements, and obtaining the frequency components of the conductance and the value of the capacitance based on multiplying the inverse matrix by a vector containing the values of the input frequency components as elements.

A carrier frequency of the input electrical signal may be greater than or equal to 20 kHz.

The method may further include generating an electrical impedance tomography image of the nerve based on the conductance and the capacitance.

According to another aspect of an example embodiment, there is provided a computer-readable recording medium having recorded thereon a program for executing a method on a computer, the method including generating an input electrical signal to be applied to a nerve, obtaining an output electrical signal based on measuring a nerve signal generated from the nerve in response to the input electrical signal, obtaining output frequency components, which are frequency components of the output electrical signal, based on converting the output electrical signal into a frequency domain, and obtaining conductance of the nerves and capacitance of the nerves based on the output frequency components.

According to another aspect of an example embodiment, there is provided an apparatus configured to analyze electrical characteristics of nerves, the apparatus including at least one processor configured to implement a signal generator configured to generate an input electrical signal to be applied to a nerve, a signal detector configured to obtain an output electrical signal based on measuring a nerve signal generated from the nerve in response to the input electrical signal, and a signal processor configured to obtain output frequency components, which are frequency components of the output electrical signal, based on converting the output electrical signal into a frequency domain and obtain conductance of the nerve and capacitance of the nerve based on the output frequency components.

The signal detector may be further configured to obtain the output electrical signal based on sampling the nerve signal at a sampling frequency, the sampling frequency being smaller than a carrier frequency of the input electrical signal.

The signal detector may be further configured to obtain the output electrical signal based on sampling the nerve signal at a sampling frequency, and the sampling frequency may be proportional to a number of the output frequency components.

The signal processor may be further configured to obtain, from a carrier frequency of the input electrical signal, the output frequency components corresponding to frequencies included in a predetermined range.

The signal processor may be further configured to obtain the output frequency components corresponding to frequencies included in a range from −3 kHz to 3 kHz from a carrier frequency of the input electrical signal.

The signal processor may be further configured to obtain frequency components of the conductance and a value of the capacitance based on the output frequency components.

The signal processor may be further configured to obtain input frequency components, which are frequency components of the input electrical signal corresponding to the output frequency components, obtain an inverse matrix of a matrix including values of the output frequency components as elements, and obtain the frequency components of the conductance and the value of the capacitance based on multiplying the inverse matrix by a vector containing the values of the input frequency components as elements.

A carrier frequency of the input electrical signal may be greater than or equal to 20 kHz.

The signal processor may be further configured to generate an electrical impedance tomography image of the nerve based on the conductance and the capacitance.

The obtaining of the output electrical signal may include obtaining the output electrical signal based on sampling the nerve signal at a sampling frequency, the sampling frequency being smaller than a carrier frequency of the input electrical signal.

### BRIEF DESCRIPTION OF THE DRAWINGS

The above and/or other aspects, features, and advantages of example embodiments will be more apparent from the following description taken in conjunction with the accompanying drawings, in which:

FIG. 1 is a diagram illustrating a nerve bundle and an apparatus for analyzing electrical characteristics of nerves according to an example embodiment;

FIG. 2 is a diagram illustrating the carrier frequency of an input electrical signal according to an example embodiment;

FIG. 3 is a diagram illustrating a control device according to an example embodiment;

FIG. 4 is a flowchart of a method of analyzing electrical characteristics of nerves according to an example embodiment;

FIG. 5 is a diagram illustrating a nerve signal according to an example embodiment;

FIG. 6 is a diagram illustrating output frequency components according to an example embodiment;

FIG. 7 is a circuit diagram for describing an example of a method of analyzing electrical characteristics of nerves according to an example embodiment;

FIG. 8 is a circuit diagram for describing an example of a method of analyzing electrical characteristics of nerves according to an example embodiment; and

FIG. 9 is a diagram illustrating electrical impedance tomography images of a nerve bundle according to an example embodiment.

### DETAILED DESCRIPTION

Reference will now be made in detail to example embodiments of which are illustrated in the accompanying drawings, wherein like reference numerals refer to like elements throughout. In this regard, the example embodiments may have different forms and should not be construed as being limited to the descriptions set forth herein. Accordingly, the example embodiments are merely described below, by referring to the figures, to explain aspects. As used herein, the term “and/or” includes any and all combinations of one or more of the associated listed items. Expressions such as “at least one of,” when preceding a list of elements, modify the entire list of elements and do not modify the individual elements of the list. For example, the expression, “at least one of a, b, and c,” should be understood as including only a, only b, only c, both a and b, both a and c, both b and c, or all of a, b, and c.

The terminology used in the example embodiments has selected general terms that are currently widely used as much as possible, but this may vary depending on the intention or precedent of a person skilled in the art or the appearance of new technologies. In addition, in certain cases, a term which is not commonly used may be selected. In such a case, the meaning of the term will be described in detail at the corresponding part in the description of the example embodiment. Therefore, the terms used in the various example embodiments should be defined based on the meanings of the terms and the descriptions provided herein.

The terms “consisting of” or “comprising” as used in the example embodiments should not be construed to include all of the various components, or various steps described in the specification. It should be construed that some steps may not be included, or may further include additional components or steps.

Hereinafter, example embodiments will be described in detail with reference to the accompanying drawings. The disclosure may, however, be embodied in many different forms and should not be construed as limited to the example embodiments set forth herein.

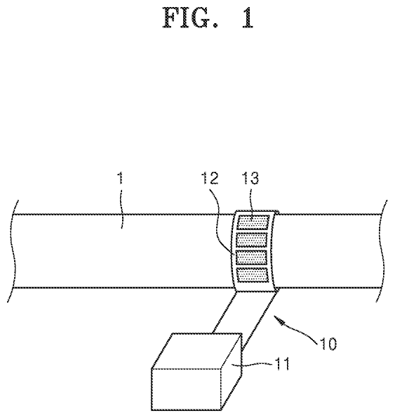

FIG. 1 is a diagram illustrating a nerve bundle and an apparatus for analyzing electrical characteristics of nerves according to an example embodiment.

Referring to FIG. 1, an apparatus 10 for analyzing electrical characteristics of nerves may include a control device 11, a cuff 12, and an electrode 13. In the apparatus 10 shown in FIG. 1, only components related to the present disclosure are shown. Therefore, it is apparent to one of ordinary skill in the art that the apparatus 10 may further include other general-purpose components in addition to the components shown in FIG. 1.

To analyze the electrical characteristics of nerves, the electrode 13 may be arranged to surround a nerve bundle 1. The apparatus 10 may include a plurality of electrodes 13, and the plurality of electrodes 13 may be arranged to surround the nerve bundle 1.

The electrode 13 may be provided on the cuff 12, and the cuff 12 may be arranged to surround the nerve bundle 1. However, embodiments are not limited thereto. For example, the electrode 13 may be arranged to surround the nerve bundle 1 without the cuff 12. The apparatus 10 may not include the cuff 12 in some example embodiments.

The control device 11 may generate an input electrical signal. An input electrical signal may be applied to the nerve bundle 1 through the electrode 13. When a nerve signal is generated from the nerve bundle 1 in response to the input electrical signal, the control device 11 may obtain an output electrical signal by measuring the nerve signal transmitted through the electrode 13.

The control device 11 may generate an electrical impedance tomography image for the nerve bundle 1 by analyzing the output electrical signal.

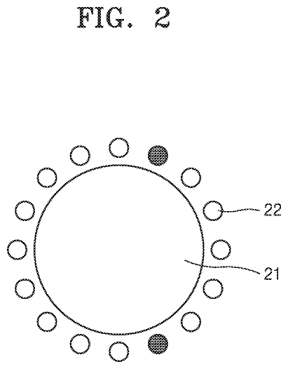

FIG. 2 is a diagram illustrating the carrier frequency of an input electrical signal according to an example embodiment.

Electrodes 22 may be arranged to surround a nerve bundle 21. In FIG. 2, 16 electrodes 22 are arranged to surround the nerve bundle 21. The number of electrodes 22 is not limited thereto. For example, 16 to 32 electrodes may be used, or other numbers of electrodes may be used.

An input electrical signal may be applied to the nerve bundle 21 through the electrodes 22. The carrier frequency of the input electrical signal may determine the number of times that electrical signals are applied to the nerve bundle 21 through the electrodes 22 per unit time.

For example, when an input electrical signal is applied at a carrier frequency of 240 kHz, electrical signals of 240 cycles may be applied to the nerve bundle 21 for 1 ms. In another example, when an input electrical signal is applied at a carrier frequency of 1 MHz, electrical signals of 1000 cycles may be applied to the nerve bundle 21 for 1 ms.

Every two of the electrodes 22 may form a pair, and a pair of electrodes may generate one electrical signal. All pairs of electrodes may generate electrical signals alternately.

For example, when 8 pairs of electrodes are used, an input electrical signal of 240 kHz may be applied to a nerve bundle as the 8 pairs of electrodes generate electrical signals of a total of 240 cycles for 1 ms. In this case, a pair of electrodes may generate 30 electrical signals. In another example, when 16 pairs of electrodes are used, an input electrical signal of 240 kHz may be applied to a nerve bundle as the 16 pairs of electrodes generate 15 electrical signals each, that is, electrical signals of a total of 240 cycles for 1 ms.

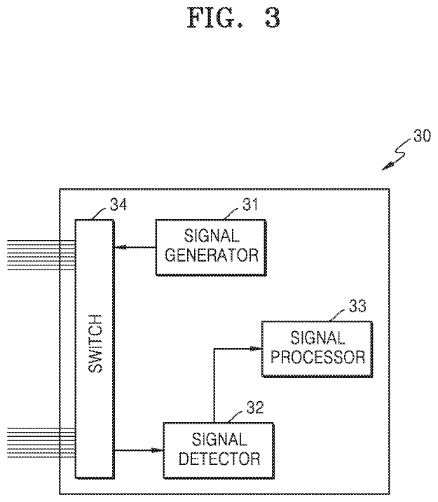

FIG. 3 is a diagram illustrating a control device according to an example embodiment.

The control device 30 may generate an input electrical signal and measure a nerve signal to obtain an output electrical signal. For example, the input electrical signal may be a current signal, whereas the output electrical signal may be a voltage signal.

The control device 30 may include a signal generator 31, a signal detector 32, and a signal processor 33. Also, the control device 30 may further include a switch 34. In the control device 30 shown in FIG. 3, only components related to the present disclosure are shown. The control device 30 may further include other general-purpose components in addition to the components shown in FIG. 3.

The signal generator 31 may generate an input electrical signal to be applied to nerves. The signal generator 31 may generate an input electrical signal of a predetermined carrier frequency.

The switch 34 may connect electrodes to the control device 30, such that the input electrical signal may be applied to the nerves through specific electrodes at a specific time. Also, the switch 34 may connect the electrodes to the control device 30, such that a nerve signal received through specific electrodes at a specific time may be measured.

The signal detector 32 may obtain an output electrical signal by measuring a nerve signal generated from the nerves in response to the input electrical signal. The signal detector 32 may obtain an output electrical signal by sampling the nerve signal at a predetermined sampling frequency.

The signal processor 33 may obtain electrical characteristics of the nerves from the output electrical signal. For example, the signal processor 33 may obtain conductance response characteristics and capacitance response characteristics of the nerves from the output electrical signal.

The signal processor 33 may be configured to generate an electrical impedance tomography image regarding a nerve bundle based on conductance response characteristics and capacitance response characteristics of the nerves. An electrical impedance tomography image may be generated by an external device connected to the control device 30.

The control device 30 may include at least one memory and at least one processor. The memory may include a random access memory (RAM), such as a dynamic random access memory (DRAM) and a static random access memory (SRAM), a read-only memory (ROM), an electrically erasable programmable read-only memory (EEPROM), a CD-ROM, a Blu-ray or another optical disc storage, a hard disk drive (HDD), a solid state drive (SSD), or a flash memory, and may further include any other external storage device that may be accessed by the control device 30. The processor may be implemented as an array of a plurality of logic gates or may be implemented as a combination of a general-purpose microprocessor and a memory in which programs that may be executed on the microprocessor are stored. Also, the processor may include a processing element for processing operations. For example, the processing element may include a logic circuit for operations. In detail, the processing element may include an operator implemented as a combination of a multiplier and an adder.

Each of the signal generator 31, the signal detector 32, and the signal processor 33 may include a processor and may be operated by the processor.

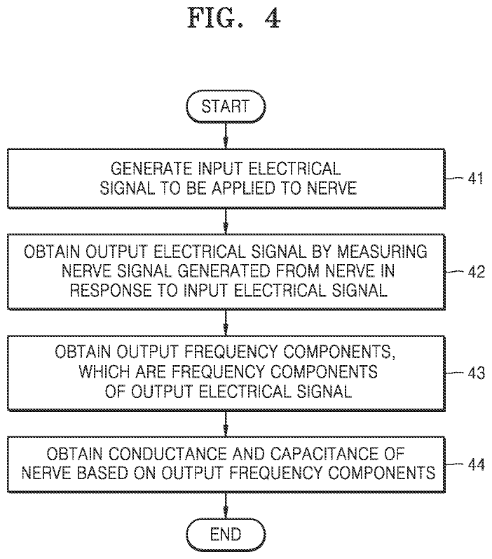

FIG. 4 is a flowchart of a method of analyzing electrical characteristics of nerves according to an example embodiment.

Referring to FIG. 4, a method of analyzing electrical characteristics of nerves includes operations performed in a time series by the apparatus 10 shown in FIG. 1, and more particularly, the control device 30 shown in FIG. 3. Therefore, it is understood that descriptions given above with respect to the apparatus 10 shown in FIG. 1 and the control device 30 shown in FIG. 3 are also applied to the method of analyzing the electrical characteristics of nerves of FIG. 4, even when the descriptions are omitted below.

In operation 41, the signal generator 31 may generate an input electrical signal to be applied to nerves. The input electrical signal may be a current signal.

The signal generator 31 may generate an input electrical signal having a carrier frequency. The range of the carrier frequency is not limited. For example, the signal generator 31 may generate an input electrical signal of a carrier frequency of 20 kHz or lower. In another example, the signal generator 31 may generate an input electrical signal of a carrier frequency of 20 kHz or higher. In another example, the signal generator 31 may generate an input electrical signal of a MHz-scale carrier frequency.

As the signal generator 31 generates an input electrical signal of a high carrier frequency of 20 kHz or higher, in analysis of electrical characteristics of nerves, time resolution may be improved and capacitance response characteristics of the nerves may be analyzed. Therefore, the precision of an electrical impedance tomography image regarding the nerves may be improved.

In operation 42, the signal detector 32 may obtain an output electrical signal by measuring a nerve signal generated from the nerves in response to the input electrical signal. The output electrical signal may be a voltage signal.

The signal detector 32 may obtain an output electrical signal by sampling the nerve signal at a sampling frequency. The sampling frequency may be lower than the carrier frequency of the input electrical signal. For example, the carrier frequency may be 240 kHz, while the sampling frequency may be 23 kHz or 29 kHz. In another example, the carrier frequency may be 1 MHz, while the sampling frequency may be 43 kHz.

The signal detector 32 or signal processor 33 may remove noise from the output electrical signal.

In operation 43, the signal processor 33 may obtain output frequency components, which are frequency components of the output electrical signal. The output electrical signal is a time domain signal, and the signal processor 33 may obtain output frequency components by converting the output electrical signal into a frequency domain signal.

The signal processor 33 may convert the output electrical signal into a frequency domain signal based on a quasiperiodic transformation. For example, the signal processor 33 may obtain output frequency components based on Equation 1 regarding quasiperiodic transformation.

$$\begin{matrix} [ Equation 1 ] \\ [ \begin{matrix}\text{?} ( {t}_{1} ) \\ \text{?} ( {t}_{2} ) \\ \text{?} ( {t}_{3} ) \\ ⋮ \\ \text{?} ( {t}_{S} )\end{matrix} ] = \\ [ \begin{matrix}1 & cos ( {ω}_{1} {t}_{1} ) & sin ( {ω}_{1} {t}_{1} ) & … & cos ( {ω}_{K} {t}_{1} ) & sin ( {ω}_{K} {t}_{1} ) \\ 1 & cos ( {ω}_{1} {t}_{2} ) & sin ( {ω}_{1} {t}_{2} ) & … & cos ( {ω}_{K} {t}_{2} ) & sin ( {ω}_{K} {t}_{2} ) \\ 1 & cos ( {ω}_{1} {t}_{3} ) & sin ( {ω}_{1} {t}_{3} ) & … & cos ( {ω}_{K} {t}_{3} ) & sin ( {ω}_{K} {t}_{3} ) \\ ⋮ & ⋮ & ⋮ & ⋱ & ⋮ & ⋮ \\ 1 & cos ( {ω}_{1} {t}_{S} ) & sin ( {ω}_{1} {t}_{S} ) & … & cos ( {ω}_{K} {t}_{S} ) & sin ( {ω}_{K} {t}_{S} )\end{matrix} ] [ \begin{matrix}{X}_{0} \\ \text{?} \\ {X}_{1}^{S} \\ ⋮ \\ \text{?} \\ \text{?}\end{matrix} ] ⇔ \\ \text{?} = {Γ}^{- 1} \cdot X \\ \text{?} \text{indicates text missing or illegible when filed}\end{matrix}$$

In Equation 1, x represents a signal in the time domain, and X represents a signal in the frequency domain. S represents the number of samplings and K represents the number of frequency components.

For example, when v represents an output electrical signal obtained by sampling the nerve signal, an output frequency components V may be obtained based on Equation 2.

Γ·v=(Γ·Γ⁻¹)·V [Equation 2]

In Equation 2, for the output frequency components V to have a solution without singularity, rows of Γ·Γ⁻¹ need to be orthogonal to each other. The signal detector 32 may perform oversampling or the signal processor 33 may performs orthogonalization after sampling, and thus the rows of Γ·Γ⁻¹ may become orthogonal to each other. For example, the signal detector 32 may perform S=6K oversampling or the signal processor 33 may perform Gram-Schmidt Orthogonalization after the signal detector 32 performs oversampling with S=4K, and thus the rows of Γ·Γ⁻¹ may be orthogonalized.

In operation 44, the signal processor 33 may obtain conductance (or resistance) and capacitance of the nerves based on the output frequency components.

The signal processor 33 may obtain conductance response characteristics (or resistance response characteristics) and capacitance response characteristics of the nerves by processing the output frequency components in the frequency domain. For example, the signal processor 33 may obtain frequency components of conductance and capacitance value.

Since the signal processor 33 analyzes electrical characteristics of nerves based on output frequency components, it is necessary to sample an output electrical signal based on the number of output frequency components needed for analysis. The sampling frequency that the signal detector 32 samples the output electrical signal may not be dependent on the carrier frequency and may be set lower than the carrier frequency. Therefore, the total computation amount for analyzing the electrical characteristics of the nerves may be reduced.

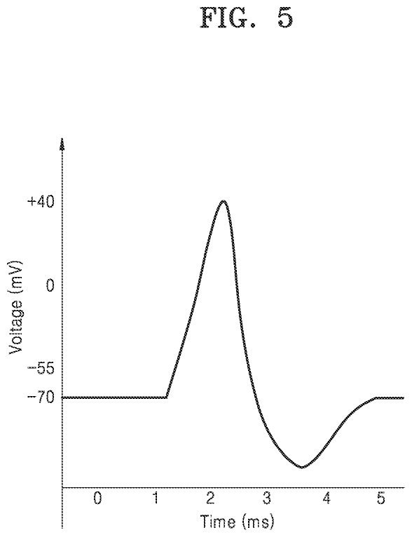

FIG. 5 is a diagram illustrating a nerve signal according to an example embodiment.

Various shape of nerve signals may be generated in nerves. FIG. 5 shows an example shape of a nerve signal.

Referring to FIG. 5, the nerve signal may be changed at a period of about 5 ms. In such a case, to analyze the electrical characteristics of nerves, it is necessary to observe the nerve signal for a device time shorter than 5 ms. For example, when 1 ms is the device time, an input electrical signal needs to have a carrier frequency at least in kHz scale.

Also, to improve the accuracy of analysis of electrical characteristics of nerves, it is necessary to apply electrical signal for a plurality of number of times during a device time. For example, when 8 pairs of electrodes 13 apply electrical signals 30 times each during a device time of 1 ms, the carrier frequency of an input electrical signal may be 240 kHz.

Considering shapes of a nerve signal and the accuracy of an analysis, the carrier frequency of the input electrical signal needs to be set high. In these example embodiments, by interpreting the electrical characteristics of nerves in the frequency domain, it is possible to analyze the electrical characteristics of the nerves by using an input electrical signal of a high carrier frequency.

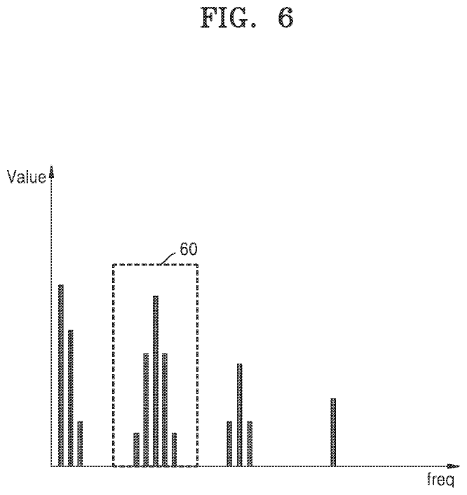

FIG. 6 is a diagram illustrating output frequency components according to an example embodiment.

Since an output electrical signal is affected by a carrier frequency ω_{c} of an input electrical signal, by considering the carrier frequency ω_{c}, a frequency range for obtaining output frequency components may be specified. Therefore, the signal processor 33 may obtain output frequency components corresponding to frequencies included in a range determined in advance from the carrier frequency ω_{c}.

When a nerve signal generated in nerves is interpreted in the frequency domain, significant frequency components of the nerve signal may be included within a specific frequency range. Therefore, to analyze the electrical characteristics of the nerves, by considering the specific frequency range, the frequency range for obtaining output frequency components may be specified. In this case, the significant frequency components of the nerve signal may refer to frequency components that may represent the nerve signal with a certain accuracy or higher.

Specifically, when the nerve signal is interpreted in the frequency domain, a limit frequency ωₘ, which is an upper limit of a specific frequency range, may be determined, and a frequency range for obtaining output frequency components may be limited by the limit frequency.

For example, when a nerve signal is interpreted in the frequency domain and the specific frequency range is from 0 kHz to 3 kHz, 3 kHz may be determined as the limit frequency, and the frequency range for obtaining output frequency components may be specified based on 3 kHz.

Therefore, the signal processor 33 may obtain output frequency components 60 corresponding to frequencies whose differences from the carrier frequency ω_{c} are less than or equal to the limit frequency ωₘ. For example, when the carrier frequency ω_{c} is 240 kHz and the limit frequency ωₘ is 3 kHz, the signal processor 33 may obtain output frequency components corresponding to frequencies included in the range from 237 kHz to 243 kHz.

The number of output frequency components obtained by the signal processor 33 may be determined by dividing the frequency range for obtaining output frequency components into unit frequency of 100 Hz. For example, when the frequency range for obtaining output frequency components is 6 kHz, by dividing 6 kHz into unit frequency of 100 Hz, the number of output frequency components may be determined as 60. The unit frequency is not limited to 100 Hz and may have different values based on the accuracy of analyzing electrical characteristics of nerves.

The sampling frequency may be determined based on the number of output frequency components. For example, the sampling frequency may be determined based on the number of output frequency components to be obtained per unit time.

For example, when the number of output frequency components to be obtained during a unit time of 10 ms is 30 and the signal detector 32 performs S=6K oversampling based on Equation 1, the sampling frequency may be determined as 18 kHz based on 100 Hz×6×30. In another example, when the number of output frequency components to be obtained during a unit time of 10 ms is 30 and the signal processor 33 performs orthogonalization after the signal detector 32 performs S=4K sampling based on Equation 1, the sampling frequency may be determined as 12 kHz based on 100 Hz×4×30.

In Equation 2, the sampling frequency may be determined to have the minimum number of common factors with the carrier frequency of an input electrical signal, such that the output frequency components V have a solution without singularity. The sampling frequency may be determined to be relatively prime with the carrier frequency in units of kHz. The sampling frequency may be determined to be a prime number in units of kHz.

For example, when the carrier frequency is 240 kHz, the sampling frequency may be determined as 23 kHz to be relatively prime with the carrier frequency in units of kHz. Since 240 and 23 are relatively prime with each other, the carrier frequency and the sampling frequency are relatively prime with each other in units of kHz.

The signal detector 32 may obtain an output electrical signal by determining the sampling frequency by using the methods described above and sampling a nerve signal at the sampling frequency.

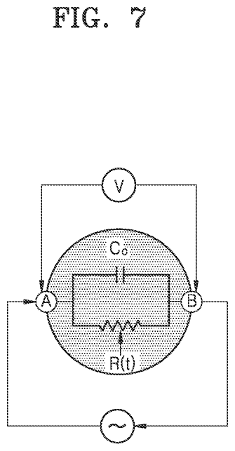

FIG. 7 is a circuit diagram for describing an example of a method of analyzing electrical characteristics of nerves according to an example embodiment.

In the circuit diagram of FIG. 7, a nerve may be modeled as a resistance R(t) and a capacitance C₀ between a node A and a node B. From the circuit diagram of FIG. 7, Equation 3, which is an equation for analyzing electrical characteristics of a nerve, may be derived.

$$i ( t ) = \sum [ g ( t ) v ( t ) + {C}_{0} \frac{dv ( t )}{dt} ] \qquad [ Equation 3 ]$$

In Equation 3, i(t) represents an input electrical signal, v(t) represents an output electrical signal, g(t) represents a conductance of a nerve and a reciprocal of a resistance R(t), and C₀ represents a capacitance of the nerve.

Equation 3 is an equation in the time domain, and, when Equation 3 is converted to an equation in the frequency domain, it may be expressed as Equation 4.

Iᵥ={G+C₀Ω}ₘ V_{c}=Vₘ┌Gᵥ:C₀┐ [Equation 4]

In Equation 4, Iᵥ represents a vector containing input frequency components that are frequency components of the input electrical signal as elements, G represents frequency components of conductance of the nerve, Gᵥ represents a vector that contains frequency components of the conductance as elements, and Vₘ represents a matrix containing the output frequency components as elements. The subscript v represents a vector, and the subscript m represents a matrix.

The signal processor 33 may obtain a frequency component G of the conductance and a capacitance value C₀ based on Equation 5.

Vₘ⁻¹·Iᵥ=┌Gᵥ:C₀┐ [Equation 5]

Specifically, the signal processor 33 may obtain input frequency components from an input electrical signal, calculate an inverse matrix Vₘ⁻¹ of a matrix including values of the output frequency components as elements, and multiplying the inverse matrix Vₘ⁻¹ by a vector Iᵥ containing values of the input frequency components, thereby obtaining frequency components Gᵥ of the conductance and the capacitance C₀.

The signal processor 33 may obtain the conductance g(t) of the nerve from the obtained frequency components G of the conductance. The signal processor 33 may generate an electrical impedance tomography image for the nerve based on the conductance g(t) and the capacitance C₀.

The conductance g(t) of the nerve varies over time, and it may be expressed as Equation 6.

$$g ( t ) = {g}_{0} + Δ g ( t ) = {g}_{0} + {\sum }_{k = - K}^{K} [ {g}_{k}^{c} cos ( k {ω}_{m} t ) + {g}_{k}^{s} sin ( k {ω}_{m} t ) ] \qquad [ Equation 6 ]$$

In Equation 6, g₀ represents the DC component of the conductance, Δg(t) represents the time-varying component of the conductance, gₖ^{c} and gₖ^{s} represent frequency components of the conductance regarding kωₘ, and ωₘ represents a limit frequency.

Output electrical signals v(t) and dv(t) may be expressed as Equation 7 and Equation 8 based on quasiperiodic transformation.

$$\begin{matrix}v ( t ) = {\sum }_{k = - K}^{K} [ {V}_{k}^{c} cos ( ( {ω}_{c} + k {ω}_{m} ) t ) + {V}_{k}^{s} sin ( ( {ω}_{c} + k {ω}_{m} ) t ) ] & [ Equation 7 ] \\ \frac{dv ( t )}{dt} = {\sum }_{k = - K}^{K} ( {ω}_{c} + k {ω}_{m} ) [ {V}_{k}^{c} cos ( ( {ω}_{c} + k {ω}_{m} ) t ) + {V}_{k}^{s} sin ( ( {ω}_{c} + k {ω}_{m} ) t ) ] & [ Equation 8 ]\end{matrix}$$

In Equations 7 and 8, Vₖ^{c} and Vₖ^{s} represent ω_{c}+kωₘ output frequency components, and ω_{c} represents the carrier frequency of the input electrical signal.

For example, when K=1 and Equation 4 is summarized by using Equations 6 to 8, it may be expressed as Equation 9.

$$[ \begin{matrix}{I}_{1}^{c} \\ {I}_{1}^{s} \\ {I}_{2}^{c} \\ {I}_{2}^{s} \\ {I}_{3}^{c} \\ {I}_{3}^{s}\end{matrix} ] = [ \begin{matrix}{V}_{1}^{c} & 0 & {V}_{2}^{c} & j ( {ω}_{c} - {ω}_{m} ) {V}_{1}^{c} \\ {V}_{1}^{s} & {V}_{2}^{s} & 0 & - j ( {ω}_{c} - {ω}_{m} ) {V}_{1}^{s} \\ {V}_{2}^{c} & {V}_{1}^{c} & {V}_{3}^{c} & j {ω}_{c} {V}_{2}^{c} \\ {V}_{2}^{s} & {V}_{3}^{s} & {V}_{1}^{s} & - j {ω}_{c} {V}_{2}^{s} \\ {V}_{3}^{c} & {V}_{2}^{c} & 0 & j ( {ω}_{c} + {ω}_{m} ) {V}_{3}^{c} \\ {V}_{3}^{s} & 0 & {V}_{2}^{s} & - j ( {ω}_{c} + {ω}_{m} ) {V}_{3}^{s}\end{matrix} ] [ \begin{matrix}{g}_{0} \\ {g}^{c} \\ {g}^{s} \\ {C}_{0}\end{matrix} ] \qquad [ Equation 9 ]$$

In Equation 9, I₁ represents an input frequency component for ω_{c}−ωₘ, I₂ represents an input frequency for ω_{c}, I₃ represents an input frequency component for ω_{c}+ωₘ, V₁ represents an output frequency component for ω_{c}−ωₘ, V₂ represents an output frequency component for ω_{c}, V₃ represents an output frequency component for ω_{c}+ωₘ, g^{c} and g^{s} represent frequency components of the conductance for ωₘ, g₀ represents a DC component of the conductance, and C₀ represents a capacitance.

For example, when K=1 and Equation 5 is summarized by using Equations 6 to 8, it may be expressed as Equation 10.

$$[ \begin{matrix}{g}_{0} \\ {g}^{c} \\ {g}^{s} \\ {C}_{0}\end{matrix} ] = {[ \begin{matrix}{V}_{1}^{c} & 0 & {V}_{2}^{c} & j ( {ω}_{c} - {ω}_{m} ) {V}_{1}^{c} \\ {V}_{1}^{s} & {V}_{2}^{s} & 0 & - j ( {ω}_{c} - {ω}_{m} ) {V}_{1}^{s} \\ {V}_{2}^{c} & {V}_{1}^{c} & {V}_{3}^{c} & j {ω}_{c} {V}_{2}^{c} \\ {V}_{2}^{s} & {V}_{3}^{s} & {V}_{1}^{s} & - j {ω}_{c} {V}_{2}^{s} \\ {V}_{3}^{c} & {V}_{2}^{c} & 0 & j ( {ω}_{c} + {ω}_{m} ) {V}_{3}^{c} \\ {V}_{3}^{s} & 0 & {V}_{2}^{s} & - j ( {ω}_{c} + {ω}_{m} ) {V}_{3}^{s}\end{matrix} ]}^{- 1} [ \begin{matrix}{I}_{1}^{c} \\ {I}_{1}^{s} \\ {I}_{2}^{c} \\ {I}_{2}^{s} \\ {I}_{3}^{c} \\ {I}_{3}^{s}\end{matrix} ] \qquad [ Equation 10 ]$$

For a nerve modeled in the circuit diagram of FIG. 7, an experiment for analyzing the electrical characteristics of the nerve was performed. In the nerve model, g₀ was set to 1.00 mS, g_{c} was set to 3.0 μS for ωₘ, and C₀ was set to 500 pF. It was assumed that there was 10 μV or 40 μV of noise in an output electrical signal. An input electrical signal was set to I₀ sin(ω_{c}t), and I₀ was set to 100 μA. In a first scenario, the signal generator 31 applied an input electrical signal having the carrier frequency of 240 kHz, and the signal detector 32 sampled a nerve signal at 29 kHz. In a second scenario, the signal generator 31 applied an input electrical signal having the carrier frequency of 1 MHz, and the signal detector 32 sampled a nerve signal at 43 kHz.

Table 1 shows the conductance and the capacitance of the nerve obtained by the signal processor 33 as a result of the experiment.

TABLE 1

| | | | Scenario 1 | | | | Scenario 2 | | | |
|---|---|---|---|---|---|---|---|---|---|---|
| | Exact | | Noise 10 μV | | Noise 40 μV | | Noise 10 μV | | Noise 40 μV | |
| g₀ | 1.00 | mS | 1.00 | mS | 1.00 | mS | 1.00 | mS | 1.00 | mS |
| g^{c} | 3.0 | μS | 2.99 | μS | 2.50 | μS | 2.54 | μS | 2.43 | μS |
| C₀ | 500 | pF | 500 | pF | 499 | pF | 500 | pF | 499 | pF |

As a result of the experiments according to the first scenario and the second scenario, it may be confirmed that the conductance and the capacitance of the nerve were obtained with high accuracy even in the presence of noise according to an example embodiment.

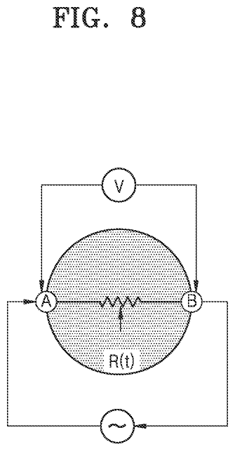

FIG. 8 is a circuit diagram for describing an example of a method of analyzing electrical characteristics of nerves according to an example embodiment.

In the circuit diagram of FIG. 8, a nerve may be modeled as the resistance R(t) between a node A and a node B.

Since the resistance R(t) varies over time, it may be expressed as Equation 11.

$$R ( t ) = {\sum }_{k = - K}^{K} [ {R}_{k}^{c} cos ( k {ω}_{m} t ) + {R}_{k}^{s} sin ( k {ω}_{m} t ) ] \qquad [ Equation 11 ]$$

For a nerve modeled in the circuit diagram of FIG. 8, an experiment for analyzing the electrical characteristics of the nerve was performed. R₀ was set to 1000Ω, R₁^{s} was set to 3Ω, and R₂^{c} was set to 2.25Ω. It was assumed that there was 10 μV or 40 μV of noise (peak-to-peak) in an output electrical signal. An input electrical signal was set to I₀ sin(ω_{c}t), and I₀ was set to 100 μA. The signal generator 31 applied an input electrical signal having the carrier frequency of 240 kHz, and the signal detector 32 sampled a nerve signal at 29 kHz.

Table 2 shows the resistance of the nerve obtained by the signal processor 33 as a result of the experiment.

TABLE 2

| | | |
|---|---|---|
| Exact | Noise 10 μV | Noise 40 μV |

| | | | | | | |
|---|---|---|---|---|---|---|
| R₀ | 1000 | Ω | 1000 | Ω | 1000 | Ω |
| R₁^{s} | 3 | Ω | 3.011 | Ω | 3.026 | Ω |
| R₂^{c} | 2.25 | Ω | 2.251 | Ω | 2.18 | Ω |

As a result of the experiment, it may be confirmed that the resistance of the nerve was obtained with high accuracy even in the presence of noise according to an example embodiment.

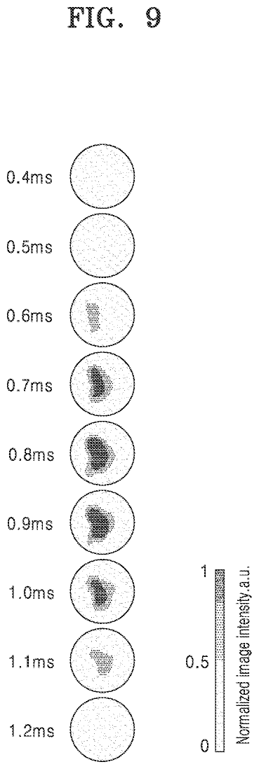

FIG. 9 is a diagram illustrating an electrical impedance tomography image for a nerve bundle according to an example embodiment.

FIG. 9 is an electrical impedance tomography image showing the electrical characteristics of nerves obtained by using the method of analyzing electrical characteristics of nerves described above with respect to a tibial fascicle.

By using a method of analyzing the electrical characteristics of nerves, the electrical characteristics of nerves may be obtained by applying a high-frequency input electrical signal, and thus electrical impedance tomography images may be obtained in real time. Also, by considering capacitance characteristics, the precision of an electrical impedance tomography image may be improved.

It should be understood that example embodiments described herein should be considered in a descriptive sense only and not for purposes of limitation. Descriptions of features or aspects within each example embodiment should typically be considered as available for other similar features or aspects in other embodiments. While example embodiments have been described with reference to the figures, it will be understood by those of ordinary skill in the art that various changes in form and details may be made therein without departing from the spirit and scope as defined by the following claims

What is claimed is:

1. A method of analyzing electrical characteristics of nerves, the method comprising:
generating an input electrical signal to be applied to a nerve;
obtaining an output electrical signal based on measuring a nerve signal generated from the nerve in response to the input electrical signal;
obtaining output frequency components, which are frequency components of the output electrical signal, based on converting the output electrical signal into a frequency domain; and
obtaining conductance of the nerves and capacitance of the nerves based on the output frequency components.

2. The method of claim 1, wherein the obtaining of the output electrical signal comprises obtaining the output electrical signal based on sampling the nerve signal at a sampling frequency, the sampling frequency being smaller than a carrier frequency of the input electrical signal.

3. The method of claim 1, wherein the obtaining of the output electrical signal comprises obtaining the output electrical signal based on sampling the nerve signal at a sampling frequency, and
wherein the sampling frequency is proportional to a number of the output frequency components.

4. The method of claim 1, wherein the obtaining of the output electrical signal comprises obtaining, from a carrier frequency of the input electrical signal, the output frequency components corresponding to frequencies included in a predetermined range.

5. The method of claim 1, wherein the obtaining of the output electrical signal comprises obtaining the output frequency components corresponding to frequencies included in a range from −3 kHz to 3 kHz from a carrier frequency of the input electrical signal.

6. The method of claim 1, wherein the obtaining of the conductance comprises obtaining frequency components of the conductance, and obtaining the capacitance comprises obtaining a value of the capacitance based on the output frequency components.

7. The method of claim 1, wherein the obtaining of the conductance and the capacitance comprises:
obtaining input frequency components, which are frequency components of the input electrical signal corresponding to the output frequency components;
obtaining an inverse matrix of a matrix comprising values of the output frequency components as elements; and
obtaining the frequency components of the conductance and the value of the capacitance based on multiplying the inverse matrix by a vector containing the values of the input frequency components as elements.

8. The method of claim 1, wherein a carrier frequency of the input electrical signal is greater than or equal to 20 kHz.

9. The method of claim 1, further comprising generating an electrical impedance tomography image of the nerve based on the conductance and the capacitance.

10. A computer-readable recording medium having recorded thereon a program for executing a method on a computer, the method comprising:
generating an input electrical signal to be applied to a nerve;
obtaining an output electrical signal based on measuring a nerve signal generated from the nerve in response to the input electrical signal;
obtaining output frequency components, which are frequency components of the output electrical signal, based on converting the output electrical signal into a frequency domain; and
obtaining conductance of the nerves and capacitance of the nerves based on the output frequency components.

11. An apparatus configured to analyze electrical characteristics of nerves, the apparatus comprising at least one processor configured to implement:
a signal generator configured to generate an input electrical signal to be applied to a nerve;
a signal detector configured to obtain an output electrical signal based on measuring a nerve signal generated from the nerve in response to the input electrical signal; and
a signal processor configured to obtain output frequency components, which are frequency components of the output electrical signal, based on converting the output electrical signal into a frequency domain and obtain conductance of the nerve and capacitance of the nerve based on the output frequency components.

12. The apparatus of claim 11, wherein the signal detector is further configured to obtain the output electrical signal based on sampling the nerve signal at a sampling frequency, the sampling frequency being smaller than a carrier frequency of the input electrical signal.

13. The apparatus of claim 11, wherein the signal detector is further configured to obtain the output electrical signal based on sampling the nerve signal at a sampling frequency, and
wherein the sampling frequency is proportional to a number of the output frequency components.

14. The apparatus of claim 11, wherein the signal processor is further configured to obtain, from a carrier frequency of the input electrical signal, the output frequency components corresponding to frequencies included in a predetermined range.

15. The apparatus of claim 11, wherein the signal processor is further configured to obtain the output frequency components corresponding to frequencies included in a range from −3 kHz to 3 kHz from a carrier frequency of the input electrical signal.

16. The apparatus of claim 11, wherein the signal processor is further configured to obtain frequency components of the conductance and a value of the capacitance based on the output frequency components.

17. The apparatus of claim 11, wherein the signal processor is further configured to:
obtain input frequency components, which are frequency components of the input electrical signal corresponding to the output frequency components;
obtain an inverse matrix of a matrix comprising values of the output frequency components as elements; and
obtain the frequency components of the conductance and the value of the capacitance based on multiplying the inverse matrix by a vector containing the values of the input frequency components as elements.

18. The apparatus of claim 11, wherein a carrier frequency of the input electrical signal is greater than or equal to 20 kHz.

19. The apparatus of claim 11, wherein the signal processor is further configured to generate an electrical impedance tomography image of the nerve based on the conductance and the capacitance.

20. The method of claim 10, wherein the obtaining of the output electrical signal comprises obtaining the output electrical signal based on sampling the nerve signal at a sampling frequency, the sampling frequency being smaller than a carrier frequency of the input electrical signal.
